(19) United States
(12) Patent Application Publication
(10) Pub. No.: US 2025/0108228 A1
(43) Pub. Date: Apr. 3, 2025

(54) MINIATURIZED C-SHAPED COIL AND SYSTEM FOR TRANSCRANIAL MAGNETIC STIMULATION

(71) Applicant: University of Southern California, Los Angeles, CA (US)

(72) Inventors: Dong Song, Walnut, CA (US); Charles Y. Liu, San Marino, CA (US); Robert Isenhart, Orange, CA (US); Wenxuan Jiang, Pasadena, CA (US)

(21) Appl. No.: 18/900,368

(22) Filed: Sep. 27, 2024

Related U.S. Application Data
(60) Provisional application No. 63/541,192, filed on Sep. 28, 2023.

(51) Int. Cl. A61N 2/00; A61N 2/02

(52) U.S. Cl. CPC A61N 2/006; A61N 2/02

(57) ABSTRACT

A system used for conducting transcranial magnetic stimulation (TMS) in a small animal is disclosed. The system may comprise: a miniaturized coil comprising C-shaped magnetic iron core having a first base and a second base, and having insulated copper wires wound around the magnetic iron core; and a stimulator that is configured to generate TMS pulses through the miniaturized coil. A method for conducting transcranial magnetic stimulation (TMS) in a small animal is also disclosed. The method may comprise: placing a miniaturized coil proximate a skull of the small animal, and generating an E-field in a brain of the small animal using the miniaturized coil.

### CROSS-REFERENCE TO RELATED APPLICATIONS

This application claims priority to U.S. Provisional Patent Application No. 63/541,192, entitled “Miniaturized C-Shaped Coil and System for Transcranial Magnetic Stimulation”, filed Sep. 28, 2023, which is hereby incorporated by reference in its entirety.

### FIELD

Disclosed herein are devices, systems, and/or methods relating to coils used for transcranial magnetic stimulation (TMS) and, more particularly, the fabrication and development of devices, systems, and/or methods associated with miniaturized TMS coils configured to enable and facilitate TMS use and focal stimulation in small animals.

### BACKGROUND

Transcranial magnetic stimulation is a non-invasive neuromodulation technique extensively used in neuromodulation, clinical applications and basic research. During TMS, a coil is placed over the scalp. A brief high-intensity current pulse passes through the coil producing a time-varying magnetic field (B-field). The B-field further induces an electric field (E-field) inside the brain, which modulates brain activities. Despite the widespread use of TMS for treating neurological and neuropsychiatric conditions such as depression, obsessive-compulsive disorder, migraine, stroke, epilepsy, schizophrenia, and autism, the underlying mechanisms of its effects remain largely unclear on humans.

However, most commercial TMS coils are designed for humans. The large geometric size of human TMS coils will cause non-focal high-intensity stimulation, which are not applicable to small animal studies. Therefore, there is a strong need for miniaturized TMS coils in neuroscience, neural engineering, and clinical research communities. Several miniaturized TMS coils have been developed for rodent studies, among which circular coil is one of the most popular designs. However, the maximum E-field of circular coils has a circular shape under the coil perimeter thus is not focal. In several recent studies, figure-eight coils were built for focal TMS in rodents. To date, figure-eight coils in conjunction with specially modified flat electrodes or probes have been used to assess brain circuitry and function. However, the figure-eight coil cannot be easily combined with standard electrophysiology since it is difficult to implant a straight penetrating electrode under the center of the coil (where maximum stimulation intensity exists) while keeping the coil close to the brain surface. Other alternative designs improved the stimulation focality using analytical models but at the same time generated E-field distributions different from those of circular or figure-eight coils, which are most commonly used in humans. Consequently, the neural responses elicited with these coils cannot be directly compared with those in human TMS studies.

Animal models are important for investigating the underlying mechanisms of TMS. However, the lack of miniaturized coils hinders the TMS studies in small animals, because available coils are designed for humans and thus incapable of focal stimulation in small animals. It is, therefore, desired to develop a miniaturized TMS coil that enables and facilitates TMS use and focal stimulation in small animals that (1) can generate focal B- and E-fields similar to those of human figure-eight coils, and (2) is compatible with standard electrophysiological recording and stimulation.

### SUMMARY

In an example embodiment, a system used for conducting transcranial magnetic stimulation (TMS) in a small animal is disclosed. The system may comprise: a miniaturized coil comprising C-shaped magnetic iron core having a first base and a second base, and having insulated copper wires wound around the magnetic iron core; and a stimulator that is configured to generate TMS pulses through the miniaturized coil.

In an example embodiment, a method for conducting transcranial magnetic stimulation (TMS) in a small animal is disclosed. The method may comprise: placing a miniaturized coil proximate a skull of the small animal, wherein the miniaturized coil comprises a dual ended magnetic iron core having a first base and a second base, wherein the first base and the second base are placed proximate the skull of the small animal, and wherein the miniaturized coil further comprises insulated copper wires wound around the magnetic iron core; and generating an E-field in a brain of the small animal using the miniaturized coil, wherein the E-field is generated by a stimulator that is configured to generate TMS pulses through the miniaturized coil.

### BRIEF DESCRIPTION OF THE DRAWINGS

These and other features and aspects of the present disclosure and methods of making the same as disclosed herein will be appreciated as the same becomes better understood by reference to the following detailed description when considered in connection with the accompanying drawings where:

FIG. 1 illustrates an example system comprising a miniaturized coil for TMS;

FIGS. 2A-2C illustrate an example miniaturized coil located proximate the brain of a rat, and B-field and E-field simulations associated therewith, in accordance with an example embodiment;

FIG. 3 is a circuit drawing of an example embodiment of a driving circuit of the TMS coil;

FIGS. 4A-4C illustrate a miniaturized coil located in proximity to a rat brain, electrodes, and measurements made in accordance with various example embodiments;

FIG. 5 is a flow chart of steps used for making an example embodiment as disclosed herein;

FIG. 6 illustrates an example array of miniaturized coils in accordance with an example embodiment as disclosed herein; and

FIG. 7 discloses stimulation patterns in accordance with various example embodiments disclosed herein.

### DESCRIPTION

Disclosed herein are miniaturized TMS coils that are specially engineered for use with small animals and that are capable of generating focal B- and E-fields similar to those of human figure-eight coils, and that are compatible with standard electrophysiological recording and stimulation. In an example embodiment, miniaturized coils as disclosed herein have C-shaped configuration to use in small animals. In an example embodiment, a miniaturized C-shaped coil comprises an insulated copper wire(s) around a C-shaped iron powder core, facilitating convenient implantation of electrophysiological recording and stimulation electrodes between the two bases of the coil. In an example embodiment, the resulting B- and E-fields are simulated with finite element modeling and further characterized with experimental measurements. In an example embodiment, the efficacy of this TMS coil in neuromodulation is validated with electrophysiological recordings of single-unit activities (SUAs), somatosensory evoked potentials (SSEPs), and MEPs before and after repetitive TMS (rTMS) in rats using single electrodes and multi-electrode arrays. Such miniaturized coils as disclosed herein and the associated experimental paradigm enabled the combination of TMS, electrophysiological recording, and electrical stimulation in rat brains. Using this paradigm, it has been for the first time observed distinct modulatory effects on SUAs, SSEPs, and MEPs with the same rTMS protocol in anesthetized rats. Thus, the miniaturized TMS coil as disclosed herein is a useful tool to investigate the neural responses as well as the underlying neurobiological mechanisms of TMS in small animal models.

In an example embodiment, a miniaturized TMS coil as disclosed herein was built by winding approximately 30 turns of insulated 23-gauge copper wire on a C-shaped magnetic core. In an example, the core was made from an iron powder toroid (T60-26, Micrometals, Anaheim CA, USA). In an example, the toroid had an outer diameter of approximately 5.2 mm, an inner diameter of approximately 8.5 mm, and a height of approximately 6.0 mm. A small gap was cut on the toroid to form the C-shaped geometry. In an example embodiment, the angle and shortest distance between the two bases of the C-shaped coil are approximately 150 degrees and 5 mm, respectively. In an example embodiment, the winding at each base of the coil was rectangular with a dimension of approximately 7 mm×4 mm. In an example embodiment, the core material comprises ferromagnetic particles coated with organic compounds to ensure electrical insulation. Such insulation makes the core tolerant of thermal aging and provides high saturation flux density with low loss. The permeability of the core material is approximately 75 times that of the air, which greatly increases the magnetic flux density of the coil. In an example embodiment, the TMS coil is dip coated with conformal silicone coating to provide extra protection and insulation. To characterize the electrical properties of the coil, the coil resistance and inductance are measured via a precision LCR meter at 1 V input voltage from 20 Hz to 100 kHz (201 points in log scale).

In an example embodiment, a TMS circuit is disclosed. The TMS circuit may comprise, for example, a stimulator for generating TMS pulses through the coil. To characterize the C-shaped TMS coil driven by the stimulator, experimental measurements of B- and E-fields were performed with a stereotaxic system in a Faraday cage. Finite element modeling of magnetic and electric fields generate by the coil were conducted.

In an example embodiment, miniaturized C-shaped coils as disclosed herein may be part of a system that includes one or more devices used to power and record use of the C-shaped coils. In an example embodiment, such system may include the TMS circuit that may be configured to generate arbitrary TMS patterns or the like, a combination of TMS as provided by the C-shaped coils was electrophysiological recording/stimulation, and/or using the TMS C-shaped coils to build a TMS array for generating spatio-temporal patterns of TMS.

The magnetic and electric fields provided by miniaturized C-shaped coils as disclosed herein were characterized with experimental measurements and finite element modeling. The efficacy of such C-shaped coils in neuromodulation was validated with electrophysiological recordings of single-unit activities (SUAs), somatosensory evoked potentials (SSEPs), and motor evoked potentials (MEPs) in rats (n=32) following repetitive TMS (rTMS; 3 min, 10 Hz). In an example embodiment, miniaturized C-shaped coils as disclosed herein may generate a maximum magnetic field of approximately 460 mT, and an electric field of approximately 7.2 V m⁻¹ in a rat brain. With subthreshold rTMS focally delivered over the sensorimotor cortex, mean firing rates of primary somatosensory and motor cortical neurons significantly increased (154±5% and 160±9% from the baseline level, respectively); MEP and SSEP amplitude significantly increased (136±9%) and decreased (74±4%), respectively. Accordingly, miniaturized C-shaped coils as disclosed herein enabled focal TMS and concurrent electrophysiological recording/stimulation at the TMS focal point. Such miniaturized C-shaped coils as disclosed herein provide a useful tool to investigate the neural responses and underlying mechanisms of TMS in small animal models. Using this paradigm, for the first time it was observed distinct modulatory effects on SUAs, SSEPs, and MEPs with the same rTMS protocol in anesthetized rats. These results suggest that multiple neurobiological mechanisms in the sensorimotor pathways were differentially modulated by rTMS. In an example embodiment, the system disclosed herein is configured to evaluate the effects of subthreshold rTMS generated by the miniature coil.

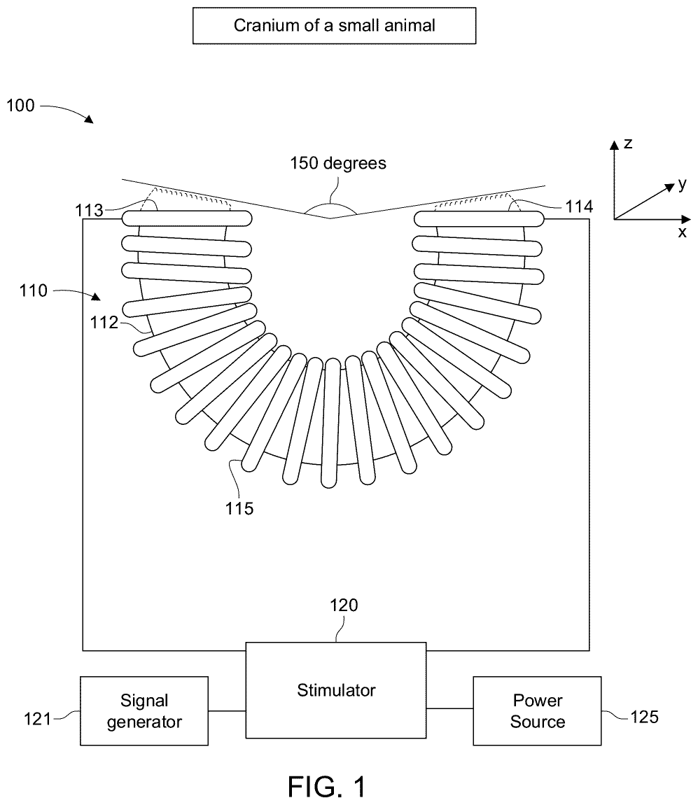

In an example embodiment, and with reference now to FIG. 1, a system 100 is disclosed for conducting transcranial magnetic stimulation (TMS) in a small animal. TMS is a non-invasive neuromodulation technique used in clinical applications and basic research. The system 100 may comprise a miniaturized coil 110. The miniaturized coil 110, in an example embodiment, comprises a magnetic iron core 112 having a first base 113 and a second base 114, and having insulated copper wires 115 wound around the magnetic iron core 112. The system 100 may further comprise a stimulator 120. In an example embodiment, the stimulator 120 is configured to generate TMS pulses through the miniaturized coil 110. In an example embodiment, the magnetic iron core 112 is a C-shaped magnetic iron core.

In an example embodiment, the magnetic iron core 112 is configured to have a center to center distance, X, between the first base 113 and second base 114 that is suitable for the cranium of a small animal, such as a rodent or rat. In one example embodiment, the distance X is between 2 mm and 10 mm, more preferably between 3 mm and 7 mm, more preferably between 4 mm and 6 mm, and in one example embodiment is 5 mm. In one example embodiment, the magnetic iron core 112 comprises first base 113 and second base 114 with planar end surfaces. In an example embodiment, the planar end surfaces are in the same plane. Stated another way, the planar end surfaces may have an angle of approximately 180 degrees between them. In this example embodiment, the magnetic iron core 112 may be more aptly described as U-shaped.

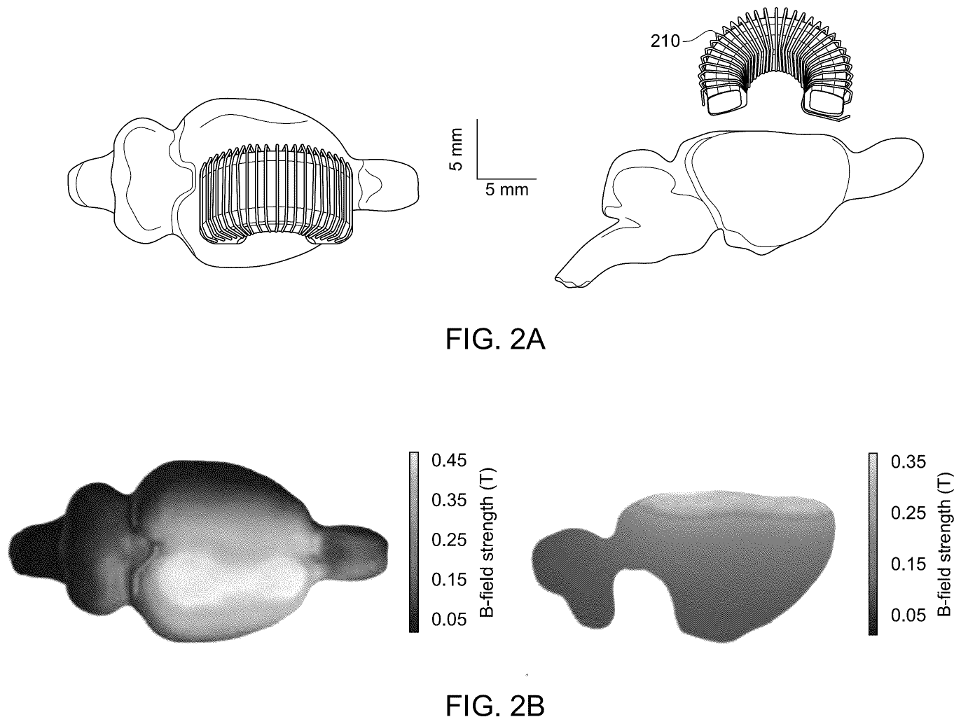

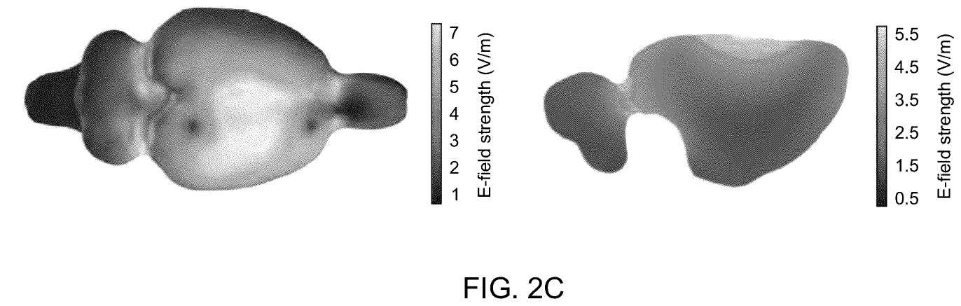

In another example embodiment, the first base 113 and second base 114 have respective planar end surfaces that have an angle between the two bases of approximately 150 degrees. See for example FIG. 1 (illustrated with dashed lines) and FIG. 2A. In various example embodiments, the angle between the two bases may be between 179 and 140 degrees, more preferably between 160 and 145 degrees, or any suitable angle. Stated differently, a first base 113 and second base 114 may be pointed inward towards each other. In this example embodiment, the magnetic iron core 112 may be described as C-shaped. Although illustrated herein as having a profile shape of a C or U, in other example embodiments, the magnetic iron core 112 is a toroid with a small gap cut to form the device, or the radius of curvature of the magnetic iron core 112 can be any suitable radius, parabolic, rectangular, and/or the like. Thus, in an example embodiment, the angle between the two bases 113, 114 may be suitable to conform to the skull of the test subject and/or may be located with the surfaces of the two bases proximate the skull of the test subject.

In an example embodiment, the magnetic iron core 112 comprises ferromagnetic particles. Moreover, the magnetic iron core 112 may comprise any suitable materials for generating suitable B-fields and E-fields. In an example embodiment, the magnetic iron core 112 is coated with an electrically insulating organic compound. For example, the magnetic iron core 112 may be dip coated with a conformal silicone coating, or any suitable coating.

In an example embodiment, the magnetic iron core 112 has a cross section that is circular, rectangular, or any suitable cross-sectional shape.

In an example embodiment, the wires 115 are made of copper. Moreover, any suitable wire material may be used for conducting a current through the wires 115. In an example embodiment, the wires 115 are insulated. In an example embodiment, the system 100 further comprises an outer layer of insulating material (not shown) disposed over the insulated copper winding(s).

In a first example embodiment, the miniaturized coil 110 comprises a single winding of wire from the first base 113 to the second base 114. In another example embodiment, miniaturized coil 110 comprises two adjacent and separate windings of copper wire at the two bases (113, 114) of the miniaturized coil 110. Moreover, in this example embodiment, the two separate windings may be configured to carry currents in opposite directions. In an example embodiment, the miniaturized coil 110 comprises a C-shaped core with two adjacent but separate coil windings at the two bases, which carry currents in opposite directions for inducing a focal E-field under the center of the coil.

In an example embodiment, the miniaturized coil 110 comprises about 50 windings of copper wire, however the number of windings can vary significantly depending on the size of the coil, and the thickness of the wire. Thus, any suitable number of windings may be used, and where possible more windings are desirable for generating larger magnetic fields.

With reference now to FIGS. 2A-2C, top and side views of a rat brain are illustrated in relation to an example miniaturized coil 210, and the B-field and E-field distributions are illustrated. The miniaturized coil 210 is illustrated in position over the scalp (proximate the skull of the rat (skull not shown)) focused on the rat brain. A brief high intensity current pulse passes through the coil producing a time-varying magnetic field (B-field). The peaks of B-field are illustrated as being underneath the two bases of the coil. See FIG. 2B. The B-field further induces an electric field (E-field) inside the brain, which modulates brain activities. In an example embodiment, the E-field distribution in the brain has a focal point located under the center of the TMS coil (See FIG. 2C). The miniaturized coil 210, in an example embodiment, is configured to deliver TMS to the M1, secondary motor cortex (M2), and S1. TMS may be useful for treating neurological and neuropsychiatric conditions such as depression, obsessive-compulsive disorder, migraine, stroke, epilepsy, schizophrenia, and autism. Animal models are important for investigating the underlying mechanisms because they allow more comprehensive electrophysiological recordings and invasive experiments that are difficult or impractical on humans. However, conventional TMS coils designed for humans are too large for use on small animals, such as rats. The large TMS coils cause non-focal high-intensity stimulation, which is not useful in small animal studies. Thus, in an example embodiment, the miniaturized TMS coil is configured to enable concurrent electrophysiological recording and stimulation at its focal point.

FIG. 2A illustrates the superior (left image) and sagittal (right image) views of coil placement relative to the 3D rat brain. FIG. 2B illustrates an example B-field distribution on the surface (left image) and sagittal section (right image) of the brain. And FIG. 2C illustrates an example E-field distribution on the surface (left image) and sagittal section (right image) of the brain.

In an example embodiment, the miniaturized coil 210 is configured to generate a maximum magnetic field of about 460 mT and an electric field of about 7.2 V m-1 in a rat brain. Moreover, the miniaturized coil 210 may be configured to generate maximum magnetic fields of any suitable value and electric fields of any suitable value. In an example embodiment, the miniaturized coil 210 is configured to generate focal magnetic fields and focal electric fields similar to those of TMS coils used in human applications. In an example embodiment, the miniaturized coil 210 is configured to deliver arbitrary stimulation pulse patterns with different intervals and amplitudes.

In an example embodiment, and with reference again to FIG. 1, the stimulator 120 is configured to receive power from a power source 125. In other example embodiments, the stimulator 120 comprises an integrated power source. In either embodiment, the power source may comprise a variable DC power source. Moreover, any suitable power source may be used provide power to the stimulator 120.

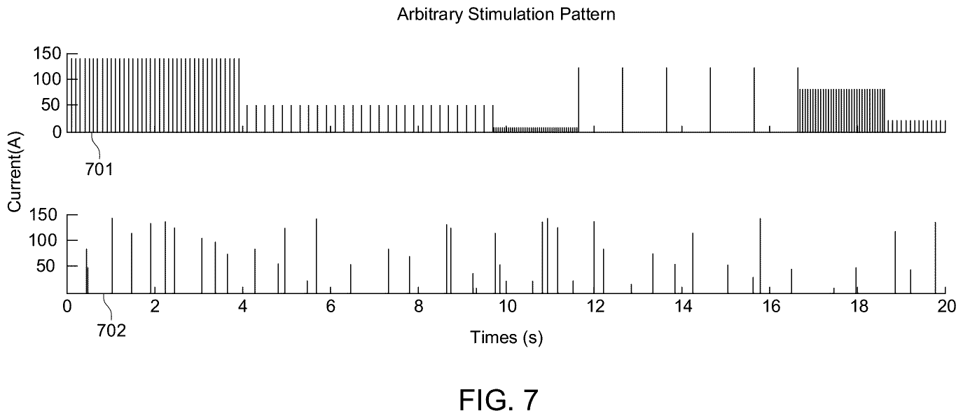

In an example embodiment, the stimulator 120 is configured to receive an input signal from a signal generator 121. In other example embodiments, the stimulator 120 comprises an integrated signal generator. In either embodiment, the signal generator may be configured to provide an input signal that is changeable. Stated another way, the signal generator 121 may generate an input signal that has any suitable shape, amplitude, and/or frequency, and/or may change that input signal over time. In one example embodiment, the signal generator 121 may generate arbitrary input signals causing the TMS coil to deliver arbitrary stimulation patterns. FIG. 7 illustrates such where the top chart 701 shows a stimulation pattern with multi-level amplitudes and frequencies and the bottom chart 702 shows a stimulation pattern with random amplitudes and intervals. In an example embodiment, the pulses are Gaussian pulses input to the stimulator 120.

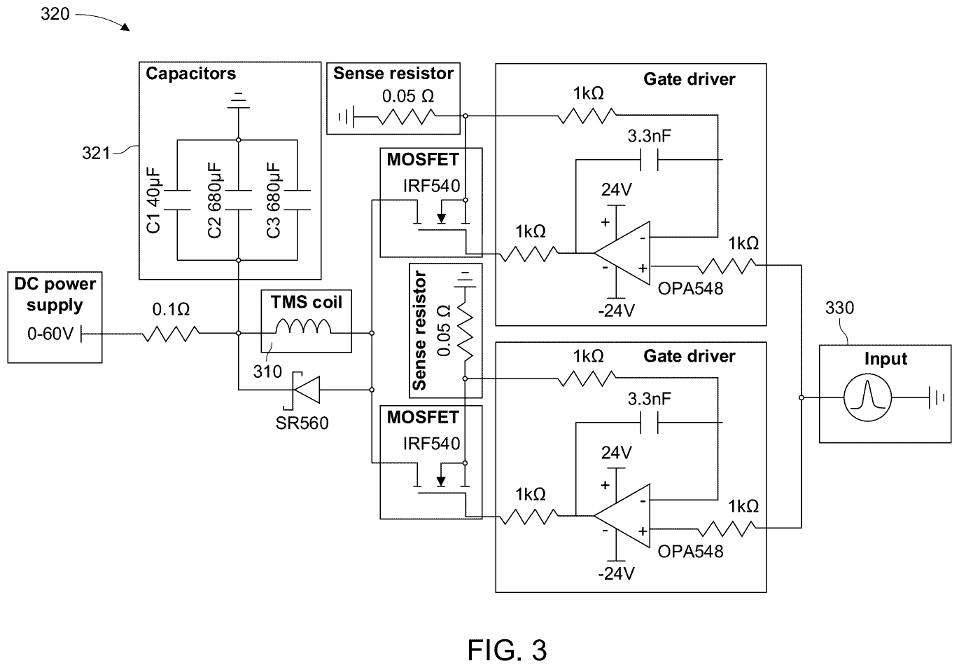

With reference now to FIGS. 1 and 3, in an example embodiment, the stimulator 120, 320 further comprises a circuit 321 (not shown in FIG. 1). In an example embodiment, the circuit 321 is a driving circuit of the TMS coil. The circuit 321 may be configured to generate TMS pulses through the coil. The circuit 321 may be configured so that an adjustment in voltage controls an amplitude of a TMS pulse produced by the coil.

In an example embodiment, the circuit 321 is illustrated showing the TMS coil 310 in relation to the circuit 321. In accordance with various example embodiments, the circuit 321 may comprise a DC power supply connected to an input of the TMS coil 310. The DC power supply may, in an example embodiment, be a variable DC power supply. The power supply may be configured to provide an adjustable voltage to control the amplitude of the TMS pulse. In an example embodiment, the maximum voltage output from the DC power supply is 60 V, though any suitable maximum voltage output may be used. Moreover, any suitable power source may be used in various example embodiments.

The circuit 321 may further comprise one or more capacitors connected to the input of the TMS coil, and configured to generate the pulses. In an example embodiment, two aluminum electrolytic capacitors and one polymer film capacitor are connected in parallel for generating the pulses. However, any suitable capacitor(s) may be used that has suitable capacitance and resistance.

In accordance with an example embodiment, a switch is connected between the output of the TMS coil and an input. The switch may comprise a pair of MOSFET transistors, or any suitable switch for controlling the charging and discharging of the capacitors. In an example embodiment, the input 330 is a waveform generator. In an example embodiment, the input 330 is configured to input to the circuit a waveform of any desired amplitude, shape, or frequency. Moreover, the input 330 (the amplitude, shape or frequency of the waveform) may be changeable from time to time. For example, the input 330 may be a sinusoidal waveform and then be switched to be a square waveform. Moreover, the input 330 may be any suitable arbitrary pulse, where ‘arbitrary’ is intended to mean that it can vary as desired for a suitable outcome. In an example embodiment, the input 330 is an arbitrary waveform generator, which can be programmed to generate monophasic waveforms as the stimulator's input.

In an example embodiment, the input 330 is converted by gate drivers to control the switch. For example, the input may be provided to two gate drivers that are configured to drive the gates of two parallel MOSFET switches. The gate drivers may comprise for example respective operational amplifiers. The input may be configured to control the on and off conditions of the two parallel MOSFET switches. When the voltage applied at the gate terminal is below the threshold voltage, the MOSFET switches are off, and the capacitors are charged up to the voltage of the DC power supply. When the voltage applied at the gate terminal is above the threshold voltage, the MOSFET switches are on, the capacitors are discharged and a current pulse is generated in the TMS coil. Moreover any suitable driving circuit may be used that receives an arbitrary or changeable input waveform for driving a current pulse of desired characteristic across a TMS coil. In an example embodiment, the two parallel MOSFET switches are used and connected in parallel to double the coil current. Connected across the TMS coil 310, in an example embodiment, is a Schottky diode to protect the switch from damage.

In an example embodiment, the gap between the two bases of the coil are configured to facilitate convenient placements of electrodes. In an example embodiment, the miniaturized TMS coils 310 facilitates implantation of electrophysiological recording and stimulation electrodes between the two bases of the coil. Thus, this disclosure enables the combination of TMS, electrophysiological recording and electrical stimulation in rat (or other small animal) brains, simultaneously. In an example embodiment, the miniaturized coil 210 is configured to enable focal TMS and concurrent electrophysical recording/stimulation at a TMS focal point.

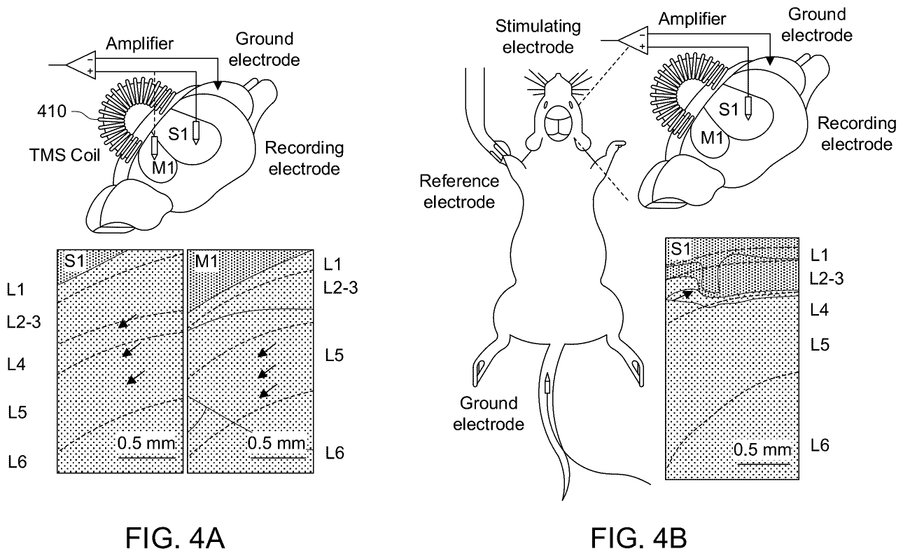

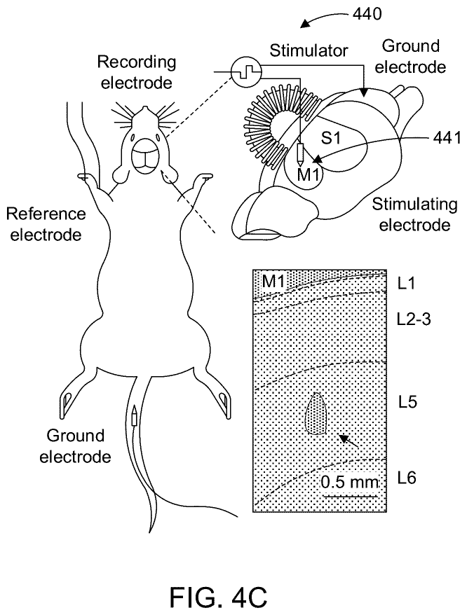

With reference now to FIGS. 4A-4C, the relative placement of the TMS coil and various electrodes is disclosed. In these embodiments, a TMS coil 410 is illustrated placed against the cranium of a rodent. The TMS coil 410 generates an E-field in the S1 and M1 regions within the brain of the rodent. Single-unit recordings can be made, for example, via electrodes implanted in the S1 and/or M1 regions. FIG. 4A illustrates an operational amplifier receiving a ground electrode and a recording electrode (located in the S1 area or located in the M1 area), for recording the resulting electrical activity at that location as a result of the TMS coil stimulation. The S1/M1 results are illustrated for single-unit activities recorded with the electrode.

FIG. 4B illustrates an operational amplifier receiving a ground electrode and a recording electrode (located in the S1 area), and the mapping of the recorded activity in the S1 area. In this set up, somatosensory evoked potentials (SSEPs) can be recorded. The recording electrode can be, in an example embodiment, a single platinum-iridium micro-electrode implanted in the S1 region. However, other suitable electrodes may be used. A reference electrode is illustrated attached to the rodent's forelimb (to stimulate the median nerve) and a ground electrode may be attached to the rodent's tail, in FIGS. 4B and 4C. In an example embodiment, a biphasic square current pulse is applied every 5 seconds to evoke SSEPs.

FIG. 4C illustrates a stimulator 440, configured to provide an electrical stimulation to a stimulating electrode 441. In this example embodiment, Motor Evoked Potentials (MEPs) are recorded using a single microelectrode implanted in the M1 region. The micro electrode and ground wire are connected to the stimulator 440.

In an example embodiment Repetitive Transcranial Magnetic Stimulation (rTMS) are delivered by providing Gaussian input pulses with, for example, a peak amplitude of 7.5 V and a standard deviation of 30 microseconds at 10 Hz.

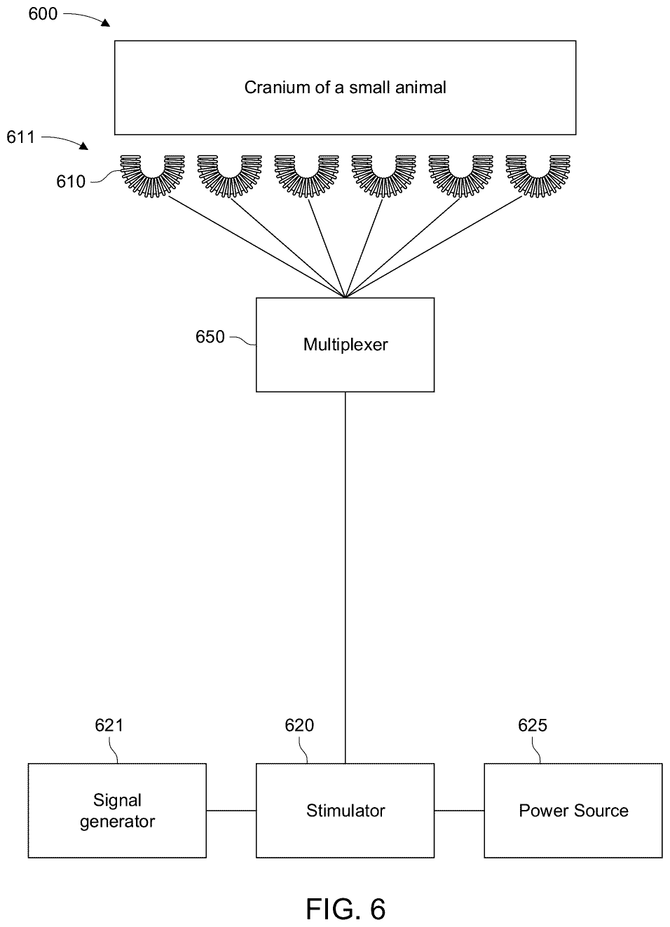

In an example embodiment, and with reference to FIG. 6, the system 600 may further comprise an array 611 of miniaturized coils 610, wherein each miniaturized coil 610 is similar to that described with respect to miniaturized coil 110. In this example embodiment, each miniaturized coil 610 is placed in various locations about the head of the test subject such that testing can be performed in multiple locations inside the head of the test subject in parallel, simultaneously, or at a minimum, without moving the miniaturized coil 610. In an example embodiment, the miniaturized coil array 611 is placed in a skull cap or helmet that holds the coils in position.

In an example embodiment, each coil of the array 611 of miniaturized coils 610 receives asynchronous signals. In an example embodiment, the stimulator 620 is configured to drive each miniaturized coil 610 independently, for example, via a multiplexer 650 or the like. Moreover, any suitable system for driving the array 611 of miniaturized coils 610 may be used.

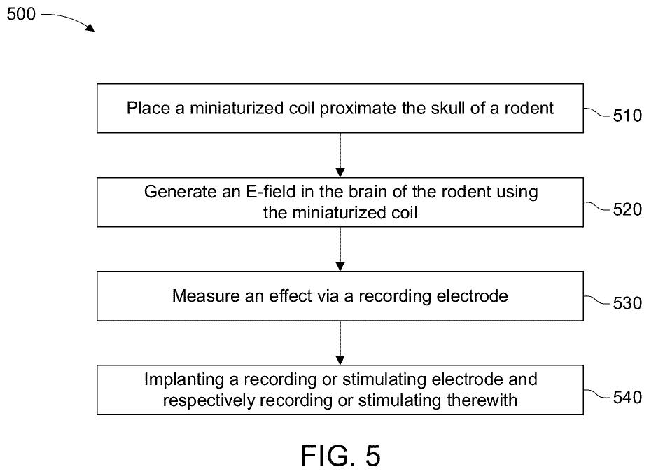

With reference now to FIG. 5, an example method 500 for using a miniaturized coil is disclosed. In an example embodiment, a miniaturized coil, similar to that described in connection with miniaturized coil 110 herein, is placed (step 510) proximate the skull of a rodent. In an example embodiment, an E-field is generated in the brain of the rodent using the miniaturized coil (step 520). In another example embodiment, the effect of the generation of the E-field may be recorded (step 530) via a recording electrode. In accordance with various example embodiments, a recording electrode and/or a stimulating electrode may be implanted in the brain, and recording and or stimulating may be performed by the respective electrode (step 540).

Exemplary embodiments of the systems, devices, and/or methods have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted except in light of the appended claims and their equivalents.

What is claimed is:

1. A system used for conducting transcranial magnetic stimulation (TMS) in a small animal comprising:
a miniaturized coil comprising C-shaped magnetic iron core having a first base and a second base, and having insulated copper wires wound around the magnetic iron core; and
a stimulator that is configured to generate TMS pulses through the miniaturized coil.

2. The system of claim 1, wherein the miniaturized coil comprises an outer layer of insulating material disposed over the insulated copper wire winding.

3. The system of claim 1, wherein magnetic iron core comprises ferromagnetic particles coated with an electrically insulating organic compound.

4. The system of claim 1, wherein the stimulator comprises a variable DC power source and a circuit, and is configured so that an adjustment in voltage controls an amplitude of a TMS pulse produced by the miniaturized coil.

5. The system of claim 1, wherein the miniaturized coil is configured to enable focal TMS and concurrent electrophysical recording/stimulation at a TMS focal point.

6. The system of claim 1, wherein the miniaturized coil is configured to generate a magnetic field and an electric field in a rat brain.

7. The system of claim 1, wherein the miniaturized coil comprises two adjacent and separate windings of copper wire at the two bases of the miniaturized coil, and wherein the windings carry currents in opposite directions.

8. The system of claim 1, wherein the miniaturized coil is configured to generate focal magnetic fields and focal electric fields similar to those of human coils.

9. The system of claim 1, wherein the miniaturized coil comprises about 50 or less windings of copper wire.

10. The system of claim 1, wherein the miniaturized coil is configured to deliver arbitrary stimulation pulse patterns with different intervals and amplitudes.

11. The system of claim 1, wherein the miniaturized coil has a distance between the two bases of between 4 mm and 6 mm.

12. A method for conducting transcranial magnetic stimulation (TMS) in a small animal comprising:
placing a miniaturized coil proximate a skull of the small animal, wherein the miniaturized coil comprises a dual ended magnetic iron core having a first base and a second base, wherein the first base and the second base are placed proximate the skull of the small animal, and wherein the miniaturized coil further comprises insulated copper wires wound around the magnetic iron core; and
generating an E-field in a brain of the small animal using the miniaturized coil, wherein the E-field is generated by a stimulator that is configured to generate TMS pulses through the miniaturized coil.

13. The method of claim 12, further comprising placing a recording electrode in the brain of the small animal.

14. The method of claim 13, further comprising measuring an effect of the E-field via the recording electrode.

15. The method of claim 12, wherein the miniaturized coil comprises two adjacent and separate windings of copper wire at the two bases of the miniaturized coil, and wherein the windings carry currents in opposite directions.

16. The method of claim 12, wherein the stimulator comprises a variable DC power source and a circuit, and is configured so that an adjustment in voltage controls an amplitude of a TMS pulse produced by the miniaturized coil.

17. The method of claim 12, wherein the miniaturized coil is configured to enable focal TMS and concurrent electrophysical recording/stimulation at a TMS focal point.

18. The method of claim 12, further comprising implanting a stimulation electrode in the brain of the small animal, and stimulating the brain using the stimulation electrode.

19. The method of claim 12, further comprising:
implanting both a stimulation electrode and a recording electrode in the brain of the small animal; and
simultaneously performing TMS, recording using the recording electrode, and
stimulating using the stimulation electrode.

20. The method of claim 19, wherein the small animal is a rodent.
